(19) United States
(12) Patent Application Publication
(10) Pub. No.: US 2026/0045358 A1
(43) Pub. Date: Feb. 12, 2026

(54) USER ACTIVITY MONITORING USING AVAILABLE WIRELESS SIGNAL STRENGTHS

(71) Applicant: ARRIS Enterprises LLC, Horsham, PA (US)

(72) Inventors: Balaji VINAYAGAM, Chennai, Tamil Nadu (IN); Dileep DEVARAJ, Kollam, Kerala (IN); Sovan ACHARYA, Bangalore, Karnataka (IN)

(21) Appl. No.: 19/101,372

(22) PCT Filed: Aug. 9, 2023
(86) PCT No.: PCT/US2023/029833

§ 371 Date: Feb. 5, 2025

Related U.S. Application Data
(60) Provisional application No. 63/398,303, filed on Aug. 16, 2022.

(51) Int. Cl. G16H 40/67; H04W 4/029; H04W 4/38; H04W 4/90; H04W 12/50

(52) U.S. Cl. CPC G16H 40/67; H04W 4/029; H04W 4/38; H04W 4/90; H04W 12/50

(57) ABSTRACT

A monitoring system can be utilized to provide remote services, for example, caregiving services, to a user. The monitoring system can perform a mapping of a site and then use this information along with sensor data to determine a status of the client user. Based on the status, the monitoring system sends a notification to a contact based on a profile configuration associated with the client user. The monitoring system can use any one or more training algorithms, such as a machine learning algorithm, to determine one or more locations of a site associated with a client user. Once these locations are determined the monitoring system is able to provide to the contact accurate and reliable information about the client user so as to improve the services and quality of experience associated with the client user.

### BACKGROUND

Wireless in general, and Wi-Fi (wireless fidelity) in particular have become ubiquitous in networking environments such that many devices that previously relied on manual readouts and displays also provide the same information over wireless technologies. This is even more important as there is a concomitant availability of software applications that run on wireless devices (such as mobile phones) that can read the data and provide useful information to the end-user, for example, via a mobile application. For example, as costs for services continue to increase, for example, healthcare services, childcare services, etc., there is an increasing desire for alternative services. While there are many individual technologies to address niche problems, given the rapid rise of connectivity technologies and the use of Artificial Intelligence techniques for predictive and analytical methods, these technologies can be confusing and difficult to configure making ubiquitous adoption of a particular technology unlikely. Additionally, services and users are increasingly requiring a visual interface with each other to permit remote communication and/or monitoring. Thus, there is a need for a more robust, cloud-based approach that provides remote capabilities including monitoring, controlling, and processing sensory data associated with a user.

### SUMMARY

Generally, there are many devices in the market that operate or behave as point solutions for specific monitoring of aspects associated with a client user. Each solution may have an associated device and an associated application that runs on the associated device. However, these solutions or technologies can require different protocols and solution-specific applications and/or devices. Further, these solutions may not be operable with other solutions or technologies already in use by a client user. Accumulating and/or analyzing the data or information from these various solutions or technologies can be daunting and thus not implementable by a client user especially when the data is particular sensitive giving rise to security and privacy concerns. According to aspects of the present disclosure there are provided novel solutions for providing one or more services associated with a client user via a remote monitoring system.

For example, providing secure, private communications between a client user and a contact, such as a trusted user, and/or specific services, such as healthcare services and/or childcare services, from a distance or remotely comes with unique challenges. To assist with monitoring of a client user remotely, a trusted user can invest in monitors with specialized sensors so as to essentially have virtual eyes and virtual ears for the monitoring of the client user. Such can require significant costs, such as associated with the installation of new activity-specific equipment. Additionally, such installations can produce false information or have system failures that require assistance from a technical administrator which adds to the cost of the system.

To overcome such costs, one or more novel aspects of the present invention utilize existing network devices within an environment associated with a client user. For example, an existing smart phone or smart watch can be utilized as a sensor device to provide sensory data associated with the client user. Such network devices can track one or more parameters associated with a client user, for example, one or more biometrics. For example, a particular network device, such as a monitoring system, can monitor one or more parameters. The one or more parameters can be indicative of one or more locations based on any of a received signal strength value change, an amplitude, a phase shift, or any combination thereof associated with one or more signals associated with any one or more network devices associated with a client user. As an example, the one or more parameters can be used in training a model so as to map one or more locations for a client user, such as one or more rooms of a premises (for example, a bedroom, a kitchen, a living room, etc. of a house associated with a client user). Any one or more algorithms can be used for training the model, for example, any of a k-nearest neighbors (KNN) algorithm, support vector machines (SVM) algorithm, any other algorithm, or any combination thereof so as to improve the mapping of one or more locations associated with the client user.

The one or more parameters or any other data associated with the client user can be sent to a contact, such as a trusted user. For example, an alert can be configured to be sent to a trusted user based on one or more parameters. The one or more parameters can be monitored, such as an RSSI value change, and mapped to an activity, location, etc. so that the trusted user is alerted based on a comparison of the one or more parameters to one or more thresholds. The trusted user can be alerted via any type of messaging, such as any of a voice message, a text message, an electronic mail message, a videoconference call, a telephone call, any other messaging, or any combination thereof.

An aspect of the present disclosure provides a monitoring device for providing a notification to a contact based on a profile configuration associated with a client user. The monitoring system comprises a memory storing one or more computer-readable instructions and a processor configured to execute the one or more computer-readable instructions to receive user location data from a client device associated with the client user, determine a location of the client user based on user location data, receive user sensor data from the client device, determine a status of the client user based on the user sensor data and the location, and provide the notification to the contact based on the profile configuration, wherein the notification comprises the status.

In an aspect of the present disclosure, the user location data comprises any of a received signal strength indicator (RSSI), an amplitude of a received signal from the client device, a phase shift of the received signal from the client device, or any combination thereof.

In an aspect of the present disclosure, the processor is further configured to execute the one or more instructions to pair the client device with the monitoring device.

In an aspect of the present disclosure, the user sensor data comprises biometric data associated with the client user.

In an aspect of the present disclosure, the processor is further configured to execute the one or more instructions to the biometric data comprises any of a movement indicator, a sleep indicator, a blood pressure, a temperature, a pulse, or any combination thereof associated with the client user.

In an aspect of the present disclosure, the providing the notification comprises the status, the location, or both.

In an aspect of the present disclosure, the processor is further configured to execute the one or more instructions to send the user location data and the user sensor data to a monitoring system, and receiving from the monitoring system one or more parameters, wherein determining the location and the status is based on the one or more parameters.

An aspect of the present disclosure provides a method for providing by a monitoring device a notification to a contact based on a profile configuration associated with the client user. The method comprises receiving user location data from a client device associated with the client user, determining a location of the client user based on the user location data, receiving user sensor data from the client device, determining a status of the client user based on the user sensor data and the location, and providing the notification to the contact based on the profile configuration, wherein the notification comprises the status.

In an aspect of the present disclosure, the method such that the user location data comprises any of a received signal strength indicator (RSSI), an amplitude of a received signal from the client device, a phase shift of the received signal from the client device, or any combination thereof.

In an aspect of the present disclosure, the method further comprising pairing the client device with the monitoring device.

In an aspect of the present disclosure, the method such that the user sensor data comprises biometric data associated with the client user.

In an aspect of the present disclosure, the method such that the biometric data comprises any of a movement indicator, a sleep indicator, a blood pressure, a temperature, a pulse, or any combination thereof associated with the client user.

In an aspect of the present disclosure, the method such that the providing the notification comprises the status, the location, or both.

In an aspect of the present disclosure, the method further comprising sending the user location data and the user sensor data to a monitoring system and receiving from the monitoring system one or more parameters, wherein determining the location and the status is based on the one or more parameters.

An aspect of the present disclosure provides a non-transitory computer-readable medium of a monitoring device storing one or more instructions for providing a notification to a contact based on a profile configuration associated with a client user. The one or more instructions when executed by a processor of the monitoring system, cause the monitoring system to perform one or more operations including the steps of the methods described above.

Thus, according to various aspects of the present disclosure described herein, it is possible to provide to a contact, such as a trusted user, a notification or alert that can comprise one or more parameters associated with a client user so as to allow the trusted user to provide immediate response and/or services to the client user. The novel solution(s) provide a monitoring system that communicates with a client device and/or one or more sensor devices to receive one or more parameters associated with a client user so as to notify a trusted user with information about the client user. The monitoring system maps one or more locations based on the one or more parameters so as to accurately identify a condition of the client user that may require notifying the trusted user. In this way, a client user can be monitored so as to receive one or more services while being remote from the trusted user and/or the monitoring system.

### BRIEF DESCRIPTION OF DRAWINGS

In the drawings, like reference numbers generally indicate identical, functionally similar, and/or structurally similar elements.

FIG. 1 is a schematic diagram of a network environment, according to one or more aspects of the present disclosure;

FIG. 2 is a more detailed block diagram illustrating various components of a network device, according to one or more aspects of the present disclosure;

FIG. 3 is an illustration of a monitoring system associated with a plurality of users within a network environment, according to one or more aspects of the present disclosure;

FIG. 4 is an illustration of a network environment for communication between a network device and a monitoring system, according to one or more aspects of the present disclosure;

FIGS. 5A, 5B, and 5C are exemplary aspects of a profile configuration for a monitoring system, according to one or more aspects of the present disclosure;

FIG. 6 illustrates exemplary signals received from a source, according to one or more aspects of the present disclosure;

FIG. 7 is a flow chart illustrating a method for providing a notification to a contact based on a profile configuration associated with a client user, according to one or more aspects of the present disclosure;

FIG. 8 illustrates a monitoring device for monitoring a client user, according to one or more aspects of the present disclosure;

FIG. 9 illustrates mapping one or more client locations associated with a client user, according to one or more aspects of the present disclosure; and

FIG. 10 illustrates user location data associated with various antennas of a monitoring device, according to one or more aspects of the present disclosure.

### DETAILED DESCRIPTION

The following detailed description is made with reference to the accompanying drawings and is provided to assist in a comprehensive understanding of various example embodiments of the present disclosure. The following description includes various details to assist in that understanding, but these are to be regarded merely as examples and not for the purpose of limiting the present disclosure as defined by the appended claims and their equivalents. The words and phrases used in the following description are merely used to enable a clear and consistent understanding of the present disclosure. In addition, descriptions of well-known structures, functions, and configurations may have been omitted for clarity and conciseness. Those of ordinary skill in the art will recognize that various changes and modifications of the examples described herein can be made without departing from the spirit and scope of the present disclosure.

Monitoring or a remote user is beneficial to provide one or more services to the user even when a contact, such as a trusted user, is remote from a user, such as a client user. For example, machine learning can be used to train a monitoring system to collect data from one or more sensing devices so as to determine an aspect or condition of the client user. The monitoring system can use the collected data to determine a location of the client user and can be compared to a threshold and a notification sent to a trusted user based on the comparison. In this way, the client user experiences an improved monitoring and the trusted user obtains key information associated with the client user even when remote from the client user.

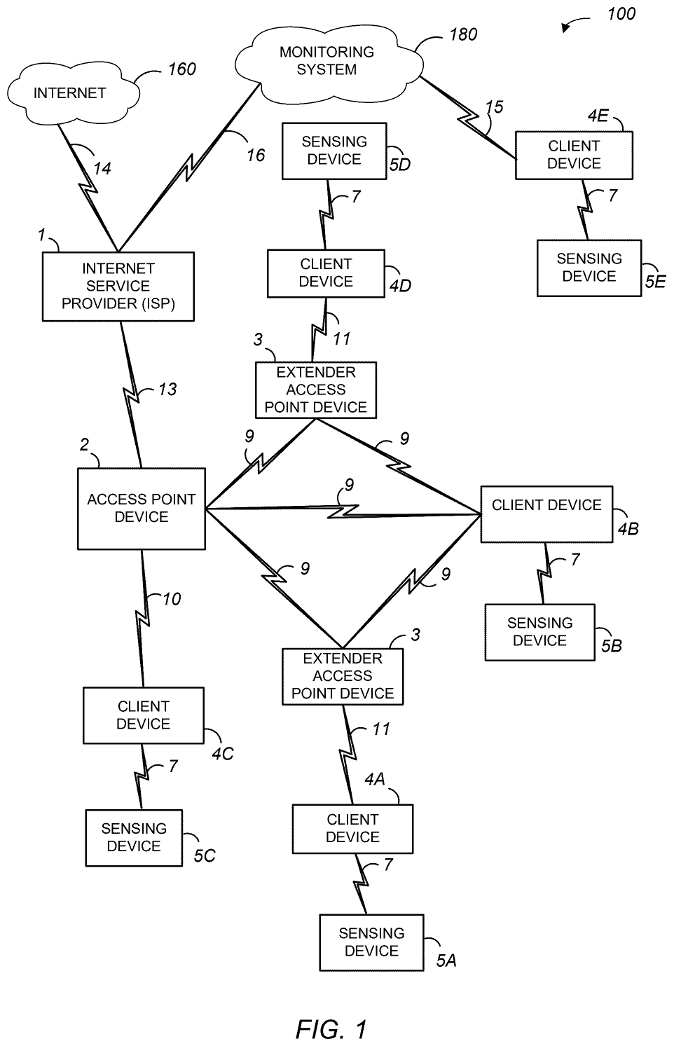

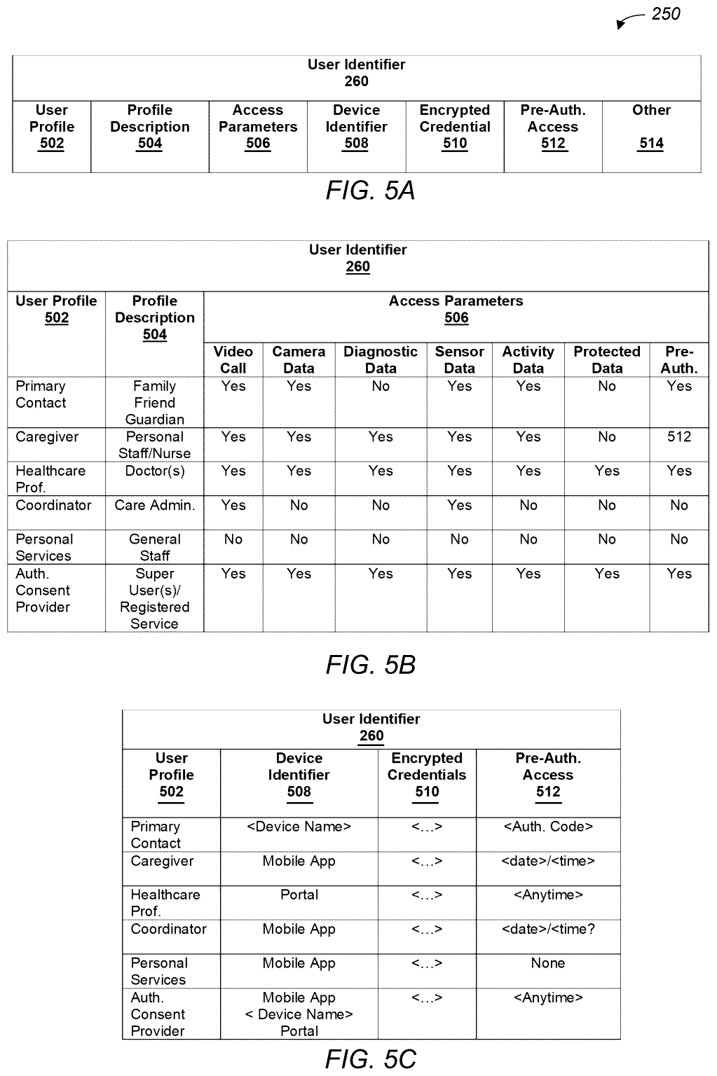

FIG. 1 is a schematic diagram of a network environment 100, according to one or more aspects of the present disclosure. For example, a secure, multi-modal, multi-protocol monitoring and communication network environment can provide for aggregation of data associated with a user, including, for example, user location data, from multiple network devices and/or sources. An example network environment can be related to a caregiving network for a user (such as a client user, for example, any of a patient, an aging-in-place user, a child, any other type of user, or any combination thereof) such that one or more aspects associated with the user (for example, biometric data, a visual interface, etc.) can be aggregated and/or monitored from multiple network devices capable of sensing the one or more conditions or aspects of the user. For example, any one or more users, such as in a trusted support network, can establish a visual interface with a particular user based on an authorization for the visual interface. Access to the aggregated and/or monitored data, including the visual interface, can be controlled based on one or more profile configurations as discussed with reference to FIGS. 5A-5C.

It should be appreciated that various example embodiments of inventive concepts disclosed herein are not limited to specific numbers or combinations of devices, and there may be one or multiple of some of the aforementioned electronic apparatuses in the network environment, which may itself consist of multiple communication networks and various known or future developed wireless connectivity technologies, protocols, devices, and the like.

As shown in FIG. 1, the main elements of the network environment 100 include a network comprising an access point device (APD) 2 connected to a network resource such as any of the Internet 160, a monitoring system 180, any other cloud storage/repository, or any combination thereof via an Internet Service Provider (ISP) 1 and also connected to different wireless devices or network devices such as one or more wireless extender access point devices 3, one or more client devices 4A-4E (collectively referred to as client device(s) 4), and one or more sensing devices 5A-5E (collectively referred to as sensing device(s) 5). The network environment 100 shown in FIG. 1 includes wireless network devices (for example, access point device 2, extender access point devices 3, client devices 4, sensing devices 5) that may be connected in one or more wireless networks (for example, private, guest, iControl, backhaul network, or Internet of things (IOT) network) within the network environment 100. Additionally, some overlap between wireless devices (for example, extender access point devices 3 and client devices 4) in the different networks can exist. That is, one or more network or wireless devices could be located in more than one network. For example, the extender access point devices 3 could be located both in a private network for providing content and information to a client device 4 and also included in a backhaul network or an iControl network.

Starting from the top of FIG. 1, the ISP 1 can be, for example, a content provider or any computer for connecting the access point device 2 to a network resource, such as Internet 160, monitoring system 180. For example, Internet 160 can be a cloud-based service that provides access to a cloud-based repository accessible via ISP 1 where the cloud-based repository comprises information associated with or an access requested by any one or more network devices of the network environment 100. The monitoring system 180 can provide monitoring, aggregation and/or controlling of data associated with a user in the network environment 100, such as data collected by one or more sensing devices 5. In one or more embodiments, the monitoring system 180 can communicate with any one or more external repositories of Internet 160 via ISP 1 or internal repositories, such as a notification repository. In one or more embodiments, any of the sensing devices 5 can be directly or indirectly coupled to the monitoring system 180 and/or any other network device, such as a monitoring device 150 discussed with reference to FIG. 8. The connection 14 between the Internet 160 and the ISP 1, the connection 16 between the monitoring system 180 and the ISP 1, the connection 15 between the monitoring system 180 and the client device 4E, and the connection 13 between the ISP 1 and the access point device 2 can be implemented using a wide area network (WAN), a virtual private network (VPN), metropolitan area networks (MANs), system area networks (SANs), a data over cable service interface specification (DOCSIS) network, a fiber optics network (e.g., FTTH (fiber to the home) or FTTX (fiber to the x), or hybrid fiber-coaxial (HFC)), a digital subscriber line (DSL), a public switched data network (PSDN), a global Telex network, or a 2G, 3G, 4G, 5G, 6G network, and/or any other network, for example.

Any of the connections 13, 14, 15, 16, or any combination thereof (collectively referred to as network connections or connections) can further include as some portion thereof a broadband mobile phone network connection, an optical network connection, or other similar connections. For example, any of the network connections can also be implemented using a fixed wireless connection that operates in accordance with, but is not limited to, 3rd Generation Partnership Project (3GPP) Long Term Evolution (LTE), 5G, or 6G protocols. It is also contemplated by the present disclosure that any of the network connections are capable of providing connections between a network device and a WAN, a LAN, a VPN, MANS, PANS, WLANs, SANS, a DOCSIS network, a fiber optics network (e.g., FTTH, FTTX, or HFC), a PSDN, a global Telex network, or a 2G, 3G, 4G, 5G, 6G network, and/or any other network, for example.

The access point device 2 can be, for example, an access point and/or a hardware electronic device that may be a combination modem and gateway that combines the functions of a modem, an access point (AP), and/or a router for providing content received from the ISP 1 to one or more network devices (for example, wireless extender access point devices 3 and client devices 4) in the network environment 100, or any combination thereof. It is also contemplated by the present disclosure that the access point device 2 can include the function of, but is not limited to, a universal plug and play (UPnP) simple network management protocol (SNMP), an Internet Protocol/Quadrature Amplitude Modulator (IP/QAM) set-top box (STB) or smart media device (SMD) that is capable of decoding audio/video content, and playing over-the-top (OTT) or multiple system operator (MSO) provided content. The access point device 2 may also be referred to as a residential gateway, a home network gateway, or a wireless access point (AP).

The connection 9 between the access point device 2 and the wireless extender access point devices 3, and client device 4B can be implemented using a wireless connection in accordance with any IEEE 802.11 Wi-Fi protocols, Bluetooth protocols, Bluetooth Low Energy (BLE), or other short range protocols that operate in accordance with a wireless technology standard for exchanging data over short distances using any licensed or unlicensed band such as the citizens broadband radio service (CBRS) band, 2.4 GHz bands, 5 GHz bands, 6 GHz bands, or 60 GHz bands. Additionally, the connection 9 can be implemented using a wireless connection that operates in accordance with, but is not limited to, RF4CE protocol, ZigBee protocol, Z-Wave protocol, or IEEE 802.15.4 protocol. It is also contemplated by the present disclosure that the connection 9 can include connections to a media over coax (MoCA) network. One or more of the connections 9 can also be a wired Ethernet connection. Any one or more of connections 9 can carry information on any of one or more channels that are available for use.

The extender access point devices 3 can be, for example, wireless hardware electronic devices such as access points (APs), extenders, repeaters, etc. used to extend the wireless network by receiving the signals transmitted by the access point device 2 and rebroadcasting the signals to, for example, client devices 4, which may be out of range of the access point device 2. The extender access point devices 3 can also receive signals from the client devices 4 and rebroadcast the signals to the access point device 2, or other client devices 4.

The connection 11 between the extender access point devices 3 and the client devices 4A and 4D are implemented through a wireless connection that operates in accordance with any IEEE 802.11 Wi-Fi protocols, Bluetooth protocols, BLE, or other short range protocols that operate in accordance with a wireless technology standard for exchanging data over short distances using any licensed or unlicensed band such as the CBRS band, 2.4 GHz bands, 5 GHz bands, 6 GHz bands, or 60 GHz bands. Additionally, the connection 11 can be implemented using a wireless connection that operates in accordance with, but is not limited to, RF4CE protocol, ZigBee protocol, Z-Wave protocol, or IEEE 802.15.4 protocol. Also, one or more of the connections 11 can be a wired Ethernet connection. Any one or more connections 11 can carry information on any one or more channels that are available for use.

The client devices 4 can be, for example, hand-held computing devices, personal computers, electronic tablets, mobile phones, smart phones, smart speakers, Internet-of-Things (IoT) devices, iControl devices, portable music players with smart capabilities capable of connecting to the Internet, cellular networks, and interconnecting with other devices via Wi-Fi and Bluetooth, or other wireless hand-held consumer electronic devices capable of executing and displaying content received through the access point device 2. Additionally, the client devices 4 can be a television (TV), an IP/QAM set-top box (STB) or a streaming media decoder (SMD) that is capable of decoding audio/video content, and playing over OTT or MSO provided content received through the access point device 2. Further, a client device 4 can be a network device that requires configuration by the access point device 2. In one or more embodiments, the client devices 4 can comprise any network device associated with a user for interacting with any type of one or more sensing devices 5. For example, the client device 4 can interact with a plurality of sensing devices 5 where each sensing device 5 senses one or more aspects associated with a user or an environment. In one or more embodiments, one or more sensing devices 5 are included within or local to (built-in) the client device 4.

One or more sensing devices 5 can connect to one or more client devices 4, for example, via a connection 7. Connection 7 can utilize any one or more protocols discussed above with respect to connection 9. Any of the one or more sensing devices 5 can comprise or be coupled to an optical instrument (such as a camera, an image capture device, any other visual user interface device, any device for capturing an image, a video, a multi-media video, or any other type of data, or a combination thereof), a biometric sensor, a biometric tracker, ambient temperature sensor, a light sensor, a humidity sensor, a motion detector (such as, an infrared motion sensor or Wi-Fi motion sensor), a facial recognition system, a medical diagnostic sensor (such as, a pulse oximeter or any other oxygen saturation sensing system, a blood pressure monitor, a temperature sensor, a glucose monitor, one or more biometric sensors, etc.), a voice recognition system, a microphone (such as, a far field voice (FFV) microphone) or other voice capture system, any other sensing device, or a combination thereof.

The connection 10 between the access point device 2 and the client device 4 is implemented through a wireless connection that operates in accordance with, but is not limited to, any IEEE 802.11 protocols. Additionally, the connection 10 between the access point device 2 and the client device 4C can also be implemented through a WAN, a LAN, a VPN, MANS, PANS, WLANs, SANS, a DOCSIS network, a fiber optics network (e.g., FTTH, FTTX, or HFC), a PSDN, a global Telex network, or a 2G, 3G, 4G, 5G, 6G network, and/or any other network, for example.

The connection 10 can also be implemented using a wireless connection in accordance with Bluetooth protocols, BLE, or other short range protocols that operate in accordance with a wireless technology standard for exchanging data over short distances using any licensed or unlicensed band such as the CBRS band, 2.4 GHz bands, 5 GHz bands, 6 GHz bands or 60 GHz bands. One or more of the connections 10 can also be a wired Ethernet connection. In one or more embodiments, any one or more client devices 4 utilize a protocol different than that of the access point device 2.

It is contemplated by the present disclosure that the monitoring system 180, the access point device 2, the extender access point devices 3, and the client devices 4 include electronic components or electronic computing devices operable to receive, transmit, process, store, and/or manage data and information associated with the network environment 100, which encompasses any suitable processing device adapted to perform computing tasks consistent with the execution of computer-readable instructions stored in a memory or a computer-readable recording medium (for example, a non-transitory computer-readable medium).

Further, any, all, or some of the computing components in the monitoring system 180, access point device 2, the extender access point devices 3, and the client devices 4 may be adapted to execute any operating system, including Linux, UNIX, Windows, MacOS, DOS, and ChromOS as well as virtual machines adapted to virtualize execution of a particular operating system, including customized and proprietary operating systems. Any one or more network devices, such as any of the monitoring system 180, the access point device 2, the extender access point devices 3, and the client devices 4, or any combination thereof are further equipped with components to facilitate communication with other computing devices or other network devices over the one or more network connections to local and wide area networks, wireless and wired networks, public and private networks, and any other communication network enabling communication in the network environment 100. Any one or more of the network devices in network environment 100 can comprise a monitoring device 150 as illustrated in FIG. 8. For example, any of a monitoring system 180, an access point device 2, a client device 4, any other network device or any combination thereof can comprise or be coupled to the monitoring device 150.

FIG. 8 illustrates a monitoring device 150, according to one or more aspects of the present disclosure. The monitoring device 150 can comprise an optical instrument or an image capture device (such as a camera 152 or any other device that can obtain one or more visuals of a client user), an audio input device (such as a microphone 154, a microphone array, a far field voice (FFV) solution, etc.), an audio output device (such as a speaker 156), a sensing device 5, and a network device 200. In one or more embodiments, any one or more components of the monitoring device 150 can be included within or external to (such as directly or indirectly connected to) the monitoring device 150. The monitoring device 150 can include any of one or more ports or receivers, for example, a Wi-Fi (such as a Wi-Fi5 (dual-band simultaneous (DBS))) port 158, a BLE port 160, an LTE port 162, an infrared (IR) blaster port 164, and IR receiver port (166), an Ethernet port 168, an HDMI-Out port 170, an HDMI-In port 172, an external power supply (such as a universal serial bus type-C(USB-C), an LED output 176, or any combination thereof. The sensing device 5 can include any one or more types of sensors, for example, as discussed with reference to FIG. 1, such as any of a power sensor, a temperature sensor, a light sensor, a humidity sensor, a motion sensor, a biometric sensor (such as a blood pressure monitor, oxygen saturation meter, pulse meter, etc.), any other type of sensor, or any combination thereof.

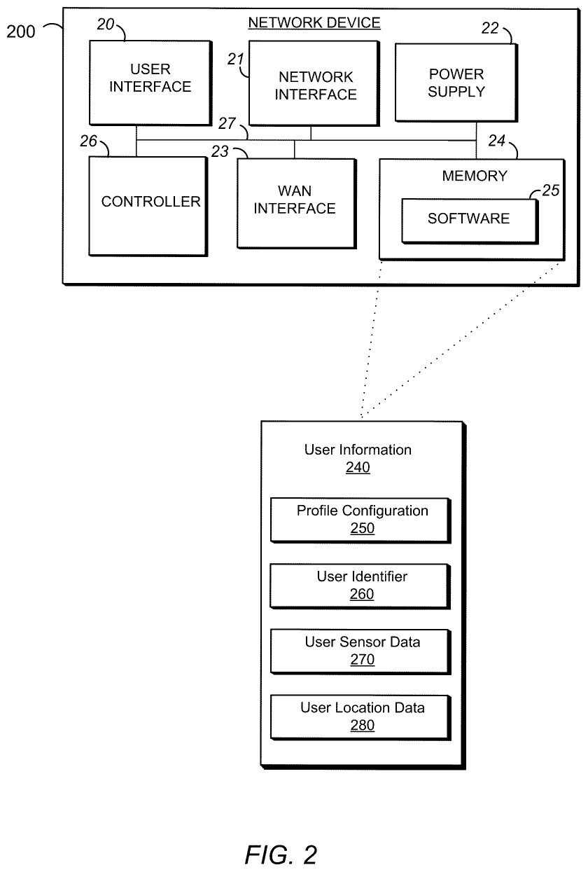

A network device, such as network device 200 discussed with reference to FIG. 2, can include software, for example, as discussed herein, to send and/or receive any of a video notification, an image (for example, an image of a client user) via camera 152, any data associated with one or more sensor devices 5, microphone 154, speaker 156, any other element, or combination thereof. Any notification can include data for display on a display device associated with the monitoring device 150 and/or a network device, for example, any of a television, a monitor, a client device 4 with display functionality connected to and/or part of the monitoring device 150, a user interface (such as user interface 20 discussed with reference to FIG. 2), or any combination thereof.

Turning back to FIG. 8, the monitoring device 150 can be connected to one or more network devices, such as any of one or more client devices 4, one or more extender access point devices 3, an access point device 2, one or more sensing devices 5, any other network device, or any combination thereof. In one or more embodiments, the monitoring device 150 pairs with a network device, such as a client device 4, so as to receive a signal from the network device, for example, a signal for determining any of an RSSI, an amplitude, a phase shift, or any combination thereof.

The monitoring device 150 can comprise any one or more elements of a network device 200. In one or more embodiments, the monitoring device 150 does not require Wi-Fi connectivity but rather can communicate with an access point device 2 using any one or more short range wireless protocols. A monitoring device 150 can include any of a BLE radio, a ZigBee radio, a LoRa radio, any other short range connectivity technology, or any combination thereof for communication to any one or more other network devices, including, but not limited to, one or more sensing devices 5.

FIG. 2 is a more detailed block diagram illustrating various components of an exemplary network device 200, such as a network device comprising a monitoring system 180, an access point device 2, an extender access point device 3, a client device 4, any other network device, or any combination thereof implemented in the network environment 100 of FIG. 1, according to one or more aspects of the present disclosure. The network device 200 can be, for example, a computer, a server, any other computer device with smart capabilities capable of connecting to the Internet, cellular networks, and interconnecting with other network devices via Wi-Fi and Bluetooth, or other wireless hand-held consumer electronic device capable of providing management and control of data, for example, a monitoring system 180, according to one or more aspects of the present disclosure. The network device 200 includes one or more internal components, such as a user interface 20, a network interface 21, a power supply 22, a controller 26, a WAN interface 23, a memory 34, and a bus 27 interconnecting the one or more elements.

The power supply 22 supplies power to the one or more internal components of the network device 200 through the internal bus 27. The power supply 22 can be a self-contained power source such as a battery pack with an interface to be powered through an electrical charger connected to an outlet (e.g., either directly or by way of another device). The power supply 22 can also include a rechargeable battery that can be detached allowing for replacement such as a nickel-cadmium (NiCd), nickel metal hydride (NiMH), a lithium-ion (Li-ion), or a lithium Polymer (Li-pol) battery.

The user interface 20 includes, but is not limited to, push buttons, a keyboard, a keypad, a liquid crystal display (LCD), a thin film transistor (TFT), a light-emitting diode (LED), a high definition (HD) or other similar display device including a display device having touch screen capabilities so as to allow interaction between a user and the network device 200, for example, for a user to enter any one or more profile configurations 250, a user identifier 260, any other information associated with a user or network device, or a combination thereof that are stored in memory 24. The network interface 20 can include, but is not limited to, various network cards, interfaces, and circuitry implemented in software and/or hardware to enable communications with and/or between the monitoring system 180, the access point device 2, an extender access point device 3, and/or a client device 4 using any one or more of the communication protocols in accordance with any one or more connections (e.g., as described with reference to FIG. 1). In one or more embodiments, the user interface 20 and/or the network interface 21 enables communications with a sensing device 5, directly or indirectly.

The memory 24 includes a single memory or one or more memories or memory locations that include, but are not limited to, a random access memory (RAM), a dynamic random access memory (DRAM) a memory buffer, a hard drive, a database, an erasable programmable read only memory (EPROM), an electrically erasable programmable read only memory (EEPROM), a read only memory (ROM), a flash memory, logic blocks of a field programmable gate array (FPGA), an optical storage system, a hard disk or any other various layers of memory hierarchy. The memory 24 can be used to store any type of instructions, software, or algorithms including software 25, for example, a monitoring application for controlling the general function and operations of the network device 200 in accordance with one or more embodiments. In one or more embodiments, memory 24 can store any one or more profile configurations 250 associated with one or more user identifiers 260 so as to provide (for example, by a monitoring application of a monitoring system 180) aggregation, monitoring, and control of data, such as user sensor data 270 received from one or more sensing devices 5, user location data 280 associated with a location of a user, or both. In one or more embodiments, any of the user information 240 can be stored locally at the network device 200, such as in memory 24, or remotely, such as at a network resource, a monitoring system 180, or both. The one or more user identifiers 260 can comprise a unique identifier associated with one or more users, one or more network devices, or both. The one or more user identifiers 260 can be associated with one or more profile configurations 250 which include information associated with one or more profiles of one or more users. The network device 200, such as a monitoring device 150, can manage and control access to data associated with the one or more user identifiers 260 based on the one or more profile configurations 250. For example, the monitoring device 150 can send a notification to a contact of a client user based on a profile configuration 250 associated with a client user, such as a client user associated with a user identifier 260.

The controller 26 controls the general operations of the network device 200 and includes, but is not limited to, a central processing unit (CPU), a hardware microprocessor, a hardware processor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software including the software 25 which can include a monitoring application in accordance with one or more embodiments. Communication between the components (for example, 20-26) of the network device 200 may be established using an internal bus 27.

The network interface 21 can include various network cards, interfaces, and circuitry implemented in software and/or hardware to enable communications with any one or more other network devices, for example, any of a client device 4, ISP 1, any other network device (for example, as described with reference to FIG. 1), or a combination thereof. The communications can utilize a visual interface connection that allows for a visual interface between two users, for example, a communication that utilizes an optical instrument (such as for a video call or for an image capture). For example, the network interface 21 can include multiple radios or sets of radios (for example, a 2.4 GHz radio, one or more 5 GHz radios, and/or a 6 GHz radio), which may also be referred to as wireless local area network (WLAN) interfaces. In one or more embodiments, one radio or set of radios (for example, 5 GHz and/or 6 GHz radio(s)) provides a backhaul connection between the wireless extender access point device 3 and the access point device 2, and optionally other wireless extender access point device(s) 3. In one or more embodiments, the monitoring system 180 is connected to or is part of the access point device 2 such that a backhaul connection is established between the monitoring system 180 and one or more wireless extender access point devices 3. Another radio or set of radios (e.g., 2.4 GHZ, 5 GHz, and/or 6 GHz radio(s)) provides a fronthaul connection between the extender access point device 3 and one or more client device(s) 4.

The wide area network (WAN) interface 23 may include various network cards, and circuitry implemented in software and/or hardware to enable communications between the access point device 2 and the ISP 1 using the wired and/or wireless protocols in accordance with connection 13 (for example, as described with reference to FIG. 1).

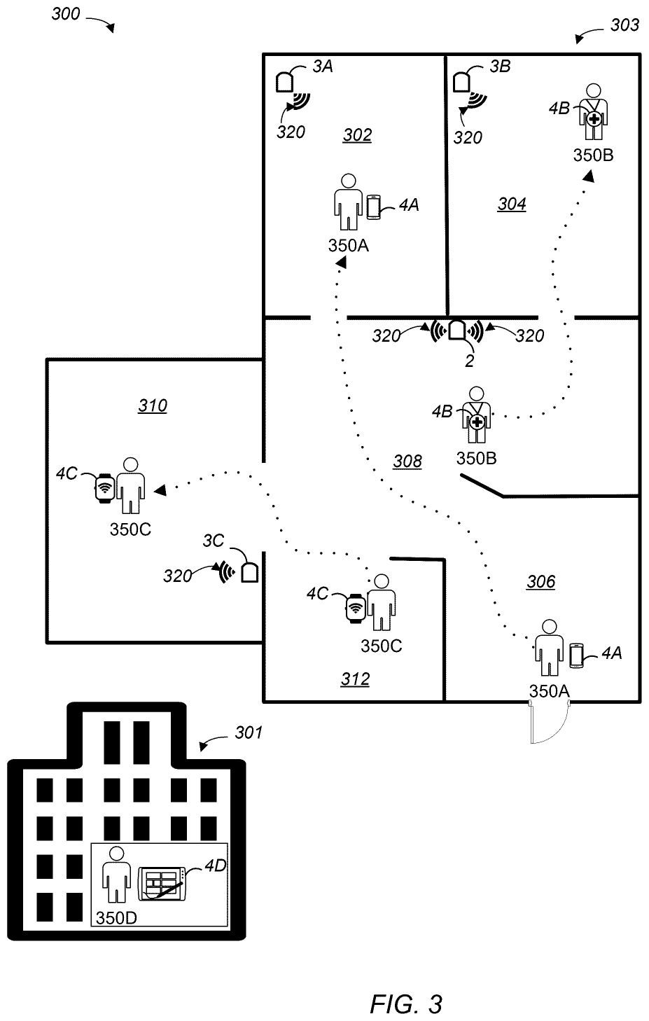

FIG. 3 is an illustration of a monitoring system, for example, an access point device 2) associated with a plurality of users within a network environment 300, according to one or more aspects of the present disclosure. The network environment 300 provides an end-to-end closed network for management, control, and access of data by one or more authorized users. The network environment 300 includes an access point device 2 that comprises a monitoring system 180, one or more client devices 4A, 4B, 4C, and 4D, and one or more extender access point devices 3. The one or more client devices 4 can include one or more sensing devices 5. One or more users 350, such as a client user 350A, a client user 350B, and a client user 350C (collectively referred to as client user(s) 350, can be disposed or at or about a site 303. An associated contact to one or more users 350 can be disposed or at or about a site 301 that is remote from the site 303.

The monitor system 180 of the access point device 2 can determine one or more locations of the site 303. The monitoring system 180 can receive one or more signals from one or more client devices 4 so as to learn or otherwise map the one or more locations within the site 303. As an example, a user 350A associated with a client device 4A, such as a smart phone, can enter the site 303 at a location 306, for example, a reception area or foyer, and transition to a location 302 that has disposed an extender access point device 3A. Based on a RSSI, an amplitude, and/or a phase shift associated with a signal received by the extender access point device 3A, the access point device 2, or both, from the client device 4A, the monitoring system 180 can map that the location 302 is a bedroom associated with the user 350A. Similarly, the monitoring system 180 can track user 350B associated with a client device 4B, such as a medical alert device, from a location 306, such as a common area, to a location 304 that includes an extender access point device 3B. Based on an RSSI, an amplitude, and/or a phase shift associated with a signal received by the extender access point device 3B, the access point device 2, or both, from the client device 4B, the monitoring system 180 can map that the location 304 is a bedroom associated with the user 350B. Similarly, the monitoring system 180 can track a user 350C from a location 312, such as a kitchen, to a location 310, such as a media room, that includes an extender access point device 3C. Based on an RSSI, an amplitude, and/or a phase shift associated with a signal (such as a signal 320 associated with one or more network devices) received by the extender access point device 3C, the monitoring system 180 can determine that the location of the user 350C based on a previous mapping of the site 303.

In one or more embodiments, the monitoring system 180 can include a training algorithm that involves mapping an RSSI, an amplitude, and/or a phase shift associated with a signal received from a network device based on a location of a client device 4 associated with a client user 350 as the client user 350 traverses multiple locations within a site 303. The layout of the site 303 can be graphed, mapped or otherwise configured so as to allow the monitoring system 180 to determine a location of the client user 350. For example, algorithms such as any of KNN, SVM, any other algorithm, or any combination thereof can be utilized to provide the mapping. In one or more embodiments, the training of an algorithm or a machine learning can comprise one or more suggestions as to placement of the access point device 2 within a site 303.

The monitoring system 180 can notify a contact, such as a trusted user 350D, via a client device 4D associated with the trusted user 350D. The notification can be based on data (a signal 320) received from an associated client device 4 and sent to the client device 4D based on information associated with the client user 350A, 350B, 350C, or any combination thereof of the client device 4A, 4B, 4C, or any combination thereof respectively, such as a profile configuration 250. The client device 4D can be associated with an emergency contact such that the client user 350D can receive notifications associated with one or more client users 350A-C. As an example, the access point device 2 that comprises a monitoring system 180 can track a client user 350 as the client user 350 transitions from a first location to a second location at a site 303 and determine based on user sensor data, location information, or both that a notification should be sent to a trusted user 350D, for example, to a client device 4D. The notification can comprise any of the user sensor data, the location information, such as a determined location of the client user 350, a request from a client device 4 associated with the client user 350 (for example, to initiate a communication), or any combination thereof.

In one or more embodiments, the monitoring system 180 tracks one or more parameters associated with a client user 350, for example, any of an activity, a biometric, any other data, or any combination thereof. The monitoring system 180 can determine to send a notification to a trusted user based on the monitoring or tracking of the one or more parameters. As an example, the monitoring system 180 can determine that no change in RSSI value associated with a single from a client device 4 associated with the client user 350 has been received within a threshold time and can send a notification to the trusted user based on the determination.

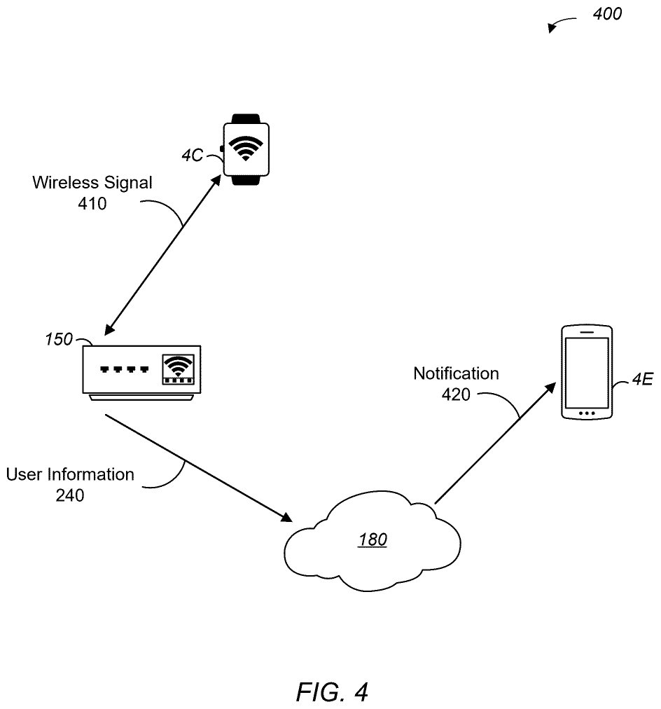

FIG. 4 is an illustration of a network environment 400 for communication between a network device and a monitoring system 180. A monitoring device 150 is communicatively coupled to a client device 4C, such as a smart watch, associated with a client user and a monitoring system 180 that is remote from the monitoring device 150. The monitoring system 180 is communicatively coupled to the client device 4E that is associated with a contact, such as a trusted user. The monitoring device 150 can determine based on a wireless signal 410 a location of a client user associated with the client device 4C. In one or more embodiments, the monitoring system 180 can receive user information 240 from the monitoring device 150 and based on this user information 240 send a notification 420 to the client device 4E associated with a trusted user. The user information can comprise the information associated with the wireless signal 410 so that the monitoring system 180 can determine a location of the client user and the notification can be based on the location. For example, if the location corresponds to a bedroom, the notification can indicate that the client user is asleep based on the location, user sensor data, or both.

FIGS. 5A, 5B, and 5C are exemplary aspects of a profile configuration 250 for a monitoring system 180, according to one or more aspects of the present disclosure. The one or more profile configurations 250 can comprise one or more parameters. For example, FIGS. 5A-5C illustrate one or more profile configurations 250 for a monitoring system 180, according to one or more aspects of the present disclosure. The one or more profile configurations 250 can be associated with a healthcare services network, a caregiver network, or any other network environment. As illustrated in FIG. 5A, the one or more parameters of a profile configuration 250 can comprise one or more user profiles 502, one or more profile descriptions 504, one or more access parameters 506, one or more device identifiers 508, one or more encrypted credentials 510, one or more pre-authorization accesses 512, any other parameters associated with a user and/or network device, or a combination thereof. Any one or more parameters associated with the user identifier 260 can be associated with a threshold that can be used by a monitory system 180 to determine to send a notification to a contact, such as a trusted user.

The one or more user profiles 502 are associated with one or more client users and/or a client device 4 associated with a client user and can include, but are not limited to, any of a primary contact, a caregiver, a healthcare professional, a coordinator, a personal service, any other type of user and/or network device, or any combination thereof. In one or more embodiments, any of the one or more user profiles 502 can be designated as a trusted user. The one or more user profiles 502 can be associated with one or more profile descriptions 504 including, but not limited to, any of a family member, friend, and/or guardian, a personal staff member or nurse, a doctor, a care administrator, a general staff member, a trusted user, any other description, or a combination thereof as illustrated in FIG. 5B. The one or more user profiles 502 can be associated with one or more access parameters 506.

The one or more access parameters 506 can include the types of data that a user or a network device associated with a corresponding user profile 502 is allowed to access, such as to view, modify, store, manage etc. In one or more embodiments, the access parameters 506 can include any alphanumeric characters, a binary value, or any other value. For example, as illustrated, a “Yes” indicates access to the data while a “No” indicates that the data is not accessible by the corresponding user profile 502. In one or more embodiments, a binary “1” or “0” could be used. The one or more access parameters 506 can include, but are not limited to, any of a video call, an image or camera data (such as from a camera), a diagnostic data (such as heart rate, blood pressure, oxygen level, weight, activity level, temperature, etc.), a sensor data, an activity data, a protected data, a pre-authorization data, any other type of data, or a combination thereof as illustrated in FIG. 5B. As an example, the pre-authorization data can indicate whether or not a pre-authorization is required to access the data by the associated user profile 502, can include a pre-authorization access 512, such as a code that indicates a pre-authorization value, that the associated user can receive responses from a client user, such as information associated with a status, a location, or both.

The creating or setting up of a profile configuration 250 can begin with assignment of roles to individuals and/or network devices (such as support users and/or) associated with a client user. Any one or more default settings could be used for any one or more of the access parameters 506. In one or more embodiments, the one or more user profiles 502 can be updated or modified dynamically.

A user identifier 260 can also be associated with a device identifier 508 such that an encrypted credential 510, a per-authorization access 512, or both can be associated with a user profile 502, a device identifier 508, or both. An encrypted credential 510 can be utilized by the monitoring system 180 to provide authorization of a request from a user associated with a user profile 502. The pre-authorization access 512 can be associated with a user profile 502 such that a user associated with the user profile 502 is pre-authorized to access user data, for example, pre-authorized to connect with a client user via a visual interface connection. A user profile 502 (that has a profile description 504) can be associated with any of a primary contact, such as a trusted user (for example, a family member, a friend, a guardian, etc.), a caregiver, such as a personal staff, a nurse, etc., a healthcare professional, such as a doctor, nurse, specialist, etc.), a coordinator (such as a care administrator), a personal services, such as general staff, an authorized consent provider, such as a super user, a registered service, etc., any other user profile, or any combination thereof.

As illustrated in FIG. 5C, for each user profile 502 associated with a user identifier 260, one or more encrypted credentials 510 and/or one or more pre-authorization accesses 512 can be associated with the user profile 502, a device identifier 508, or both. In one or more embodiments, a device identifier 508 can be associated with a device name, a mobile application, a portal, any other type of device or resource, or any combination thereof. In one or more embodiments, the pre-authorization access 512 can be indicative of an authorization code or time period, such as a date and/or time, that pre-authorization is permitted.

While FIGS. 5A-5C illustrate one or more profile configurations 250 associated with a healthcare services network, the present disclosure contemplates that the one or more profile configurations 250 can be associated with any type of network. Additionally, the present disclosure contemplates that any one or more user profiles 502, one or more profile descriptions 504, one or more access parameters 506, one or more scheduling parameters, or any combination thereof can be added or deleted based on a particular network environment, including dynamically.

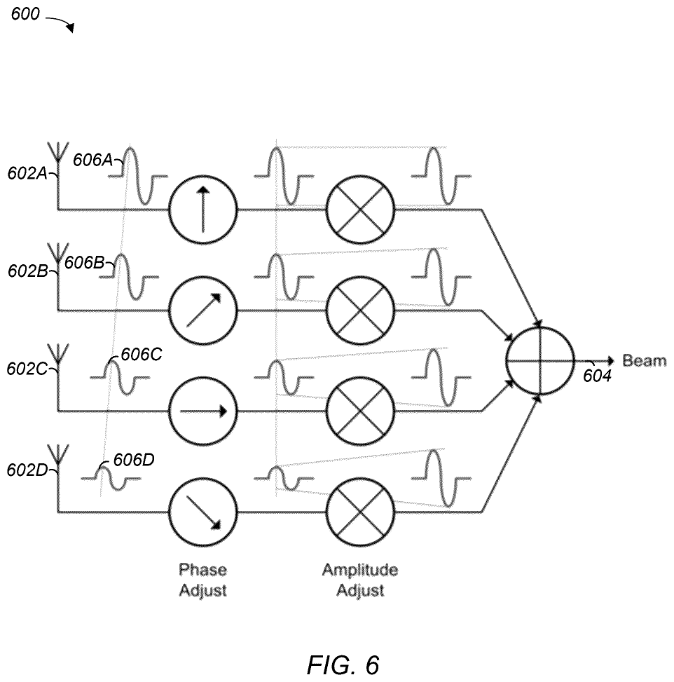

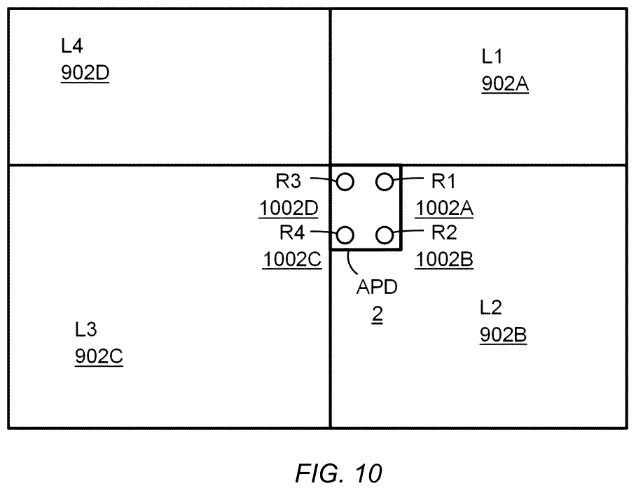

FIG. 6 illustrates exemplary signals received from a source 602, according to one or more aspects of the present disclosure. One or more network devices within a network environment can comprise one or more antennas. FIG. 6 illustrates a beam forming mechanism that uses amplitude phase-shift and RSSI value difference values at each antenna 602, for example, antennas 602A, 602B, 602C, and 602D (collectively referred to as antenna 602), to form a beam 604. The beam 604 is generated using the property of interference of multiple waves, for example, wave 606A, wave 606B, wave 606C, and wave 606D, collectively referred to as waves 606. If the multiple waves 606 interfere with each other at “in-phase”, the amplitude of the interfered waves gets bigger (referred to as constructive interference). If the multiple waves 606 propagating in 2D or 3D spaces, the resulting interference would show a specific pattern in which some part of the spaces shows constructive interference and some other parts show destructive interference. The part performing constructive interference forms a beam pointing to a specific direction. The client user at each and every location at a premise or site will give a different value with respect to R1, R2, r3, and R4 as illustrated in FIG. 10. This phase-shift, amplitude values, received at each antenna 602A-D (or R1, R2, R3, and R4 as illustrated in FIG. 10) to map and/or plot locations L1, L2, L3 and L4 at the premise or site as illustrated in FIG. 10.

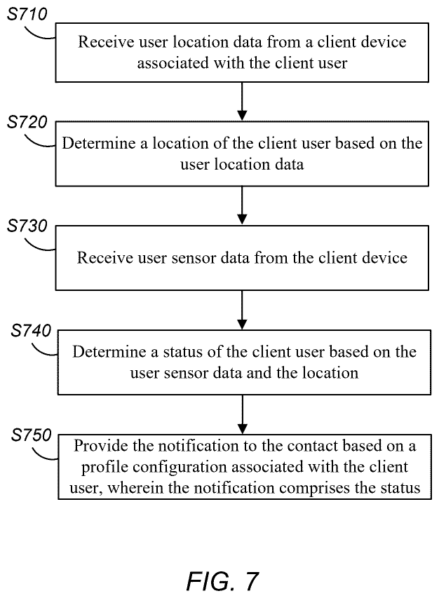

FIG. 7 is a flow chart illustrating a method for providing a notification to a contact based on a profile configuration associated with a client user, according to one or more aspects of the present disclosure. A monitoring device 150 may be programmed with one or more instructions such as a monitoring application that when executed by a processor or controller causes the monitoring device 150 to provide a notification to a contact based on a profile configuration associated with a client user. In FIG. 7, it is assumed that any one or more of the network devices include their respective controllers and their respective software stored in their respective memories, as discussed above in connection with FIGS. 1-6 and 8-10, which when executed by their respective controllers perform the functions and operations in accordance with the example embodiments of the present disclosure (for example, including receiving user sensor data from one or more sensing devices 5).

The monitoring device 150 comprises a controller 26 that executes one or more computer-readable instructions, stored on a memory 24, that when executed perform one or more of the operations of steps S710-S750. The monitoring device 150 can comprise one or more software 25, for example, a software 25. While the steps S710-S750 are presented in a certain order, the present disclosure contemplates that any one or more steps can be performed simultaneously, substantially simultaneously, repeatedly, in any order or not at all (omitted). The monitoring device 150 can be coupled to or be included within a monitoring system 180.

At step S710, the monitoring device 150 receives user location data from a client device associated with the client user. As an example, the monitoring device 150 can be located at a premise or site associated with the client user, for example, as, as part of, or included within any of a set-top box, an access point device 2, any other network device, or any combination thereof. As another example, the monitoring device 150 can be included within a monitoring system 180 that is located remote from the client user as illustrated in FIG. 1, such that an access point device 2 transmits or sends the user location data via an ISP 1 to the monitoring device 150. In one or more embodiments, the monitoring device 150 is paired with the client device. The monitoring device 150 can receive user location data as the client user transitions throughout the premise. The user location data can comprise any of an RSSI, an amplitude of a received signal from the client device, a phase shift of the received signal from the client device, or any combination thereof.

At step S720, the monitoring device 150 determines a location of the client user based on the user location data from step S710. In one or more embodiments, the monitoring device 150 can determine the location of the client user using the user location data as an input to a machine learning algorithm. As an example, the monitoring device 150 can send the user location data to a monitoring system 180 (whether remote from or local to the monitoring device 150) and can determine the location of the client user based on information received from the monitoring system 180, such as the location and/or other data.

At step S730, the monitoring device 150 can receive user sensor data from the client device. For example, the client device can be or be connected to a sensing device that monitors or detects user sensor data associated with the client user, such as a biometric sensing device that monitors and/or detects biometric data associated with the user. The biometric data comprises any of a movement indicator, a sleep indicator, a blood pressure, a temperature, a pulse, or any combination thereof associated with the client user. In one or more embodiments, the user sensor data can be sent to a monitoring system 180. In response, the monitoring system 180 sends the monitoring device 150 one or more parameters

At step S740, the monitoring device 150 determines a status of the client user based on the user sensor data and the location determined at step S720. In one or more embodiments, the status and the location are determined based on the one or more parameters received from the monitoring system 180 as discussed with reference to step S730. The status of the client user can indicate a condition of the user, such as any of asleep, awake, active, non-active, exercising, in distress, normal, abnormal, any other condition, or any combination thereof.

At step S750, the monitoring device 150 can provide a notification to a contact based on the status determined at step S740. The contact can be determined based on a profile configuration associated with the client user. The notification can comprise the status, the location, the user sensor data, any other data, or any combination thereof.

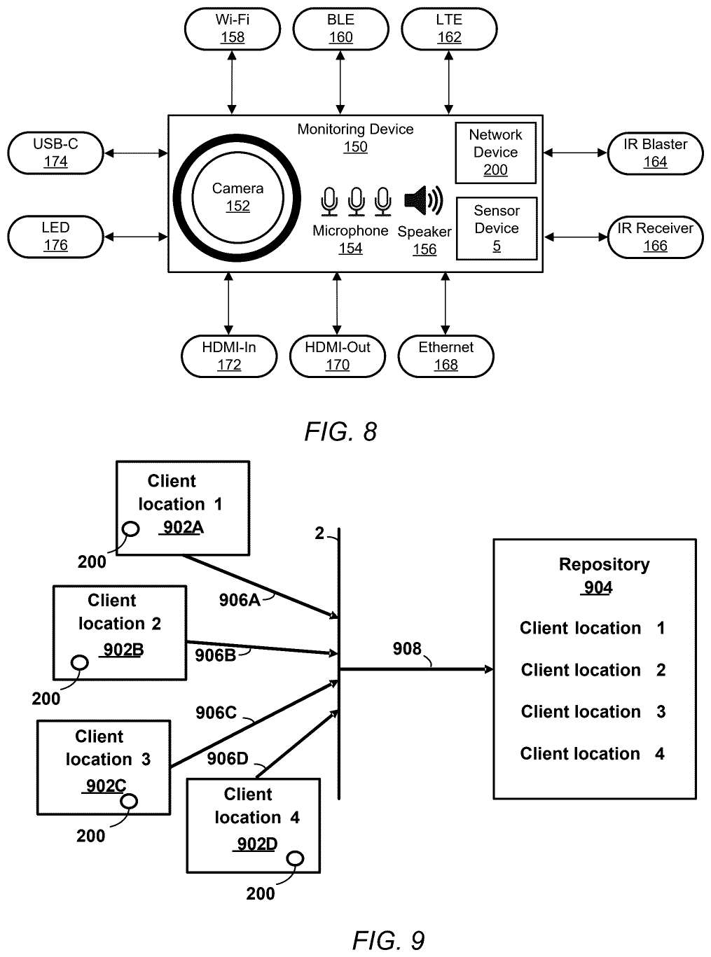

FIG. 9 illustrates mapping one or more client locations 902 associated with a client user, according to one or more aspects of the present disclosure. One or more network devices 200 can be transited throughout a site, for example, from a client location 1 902A, to a client location 2 902B, to a client location 3 902C to a client location 4 902D, collectively referred to as client location(s) 902. At each client location 902, the network device 200 sends a communication or signal to a monitoring system 180, for example, an access point device 2. The access point device 2 collects the communications (for example, signal 906A associated with client location 1 902A, signal 906B association with client location 2 902B, signal 906C associated with client location 3 902C, and signal 906D associated with client location 4 902D) and sends an instruction 908 to a repository 904 to store each respective signal 906. At each client location 902, a client device 4 can be utilized to confirm the location and to identify the location within the site. For example, the client location 1 902A can be mapped as a bedroom, a client location 2 902B can be mapped as a kitchen, a client location 3 902C can be mapped as a media/common room, and a client location 4 902D can be mapped as a bathroom. The repository 904 can be located remote from access point device 2, for example, in the cloud, such as accessible via an ISP 1, and/or local to the access point device 2. The client locations 902 can be stored in the repository 904 so that the monitoring system 180 can map a site, such as a home, an assisted living center, a facility, or any other site that requires tracking of a client user. For example, the monitoring system 180 utilizes a training algorithm to map a site that comprises the one or more client locations 902. As another example, the monitoring system 180 can determine whether to send a notification to a trusted user based on any of a RSSI, an amplitude, a phase shift, or any combination thereof associated with any one or more signals 906.

FIG. 10 illustrates user location data associated with various antennas of a monitoring device, according to one or more aspects of the present disclosure. LI corresponds to client location 1 902A, L2 corresponds to client location 2 902B, L3 corresponds to client location 3 902C, and L4 corresponds to client location 4 902D. An access point device 2 can be located at L2. The access point device 2 can comprise one or more antennas, such as antenna R1 1002A, antenna R2 1002B, antenna R3 1002D, and antenna R4 1002C, collectively referred to as antenna(s) 1002. The RSSI, the amplitude and/or the phase shift values received at each of the antennas 1002 (for example, antennas R1, R2, R3, and R4) can be mapped for each client location 902 (for example, L1, L2, L3, and L4) during training of the monitoring system 180, for example, as illustrated by TABLE 1.

TABLE 1

| Location | R1 | R2 | R3 | R4 |
|---|---|---|---|---|
| L1 | 1 | 3 | 2 | 4 |
| L2 | 3 | 1 | 4 | 2 |
| L3 | 4 | 2 | 3 | 1 |
| L4 | 3 | 4 | 1 | 2 |

According to one or more example embodiments of inventive concepts disclosed herein, there are provided novel solutions for monitoring, tracking, mapping and providing a notification based on a client user for a site. The novel solutions according to example embodiments of inventive concepts disclosed herein provide features that improve the monitoring, tracking, and identifying a client user within a site.

Each of the elements of the present invention may be configured by implementing dedicated hardware or a software program on a memory controlling a processor to perform the functions of any of the components or combinations thereof. Any of the components may be implemented as a CPU or other processor reading and executing a software program from a recording medium such as a hard disk or a semiconductor memory, for example. The processes disclosed above constitute examples of algorithms that can be affected by software, applications (apps, or mobile apps), or computer programs. The software, applications, computer programs or algorithms can be stored on a non-transitory computer-readable medium for instructing a computer, such as a processor in an electronic apparatus, to execute the methods or algorithms described herein and shown in the drawing figures. The software and computer programs, which can also be referred to as programs, applications, components, or code, include machine instructions for a programmable processor, and can be implemented in a high-level procedural language, an object-oriented programming language, a functional programming language, a logical programming language, or an assembly language or machine language.

The term “non-transitory computer-readable medium” refers to any computer program product, apparatus or device, such as a magnetic disk, optical disk, solid-state storage device (SSD), memory, and programmable logic devices (PLDs), used to provide machine instructions or data to a programmable data processor, including a computer-readable medium that receives machine instructions as a computer-readable signal. By way of example, a computer-readable medium can comprise DRAM, RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, includes compact disc (CD), laser disc, optical disc, digital versatile disc (DVD), floppy disk and Blu-ray disc. Combinations of the above are also included within the scope of computer-readable media.

The word “comprise” or a derivative thereof, when used in a claim, is used in a nonexclusive sense that is not intended to exclude the presence of other elements or steps in a claimed structure or method. As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise. Use of the phrases “capable of,” “configured to,” or “operable to” in one or more embodiments refers to some apparatus, logic, hardware, and/or element designed in such a way to enable use thereof in a specified manner.

While the principles of the inventive concepts have been described above in connection with specific devices, apparatuses, systems, algorithms, programs and/or methods, it is to be clearly understood that this description is made only by way of example and not as limitation. The above description illustrates various example embodiments along with examples of how aspects of particular embodiments may be implemented and are presented to illustrate the flexibility and advantages of particular embodiments as defined by the following claims, and should not be deemed to be the only embodiments. One of ordinary skill in the art will appreciate that based on the above disclosure and the following claims, other arrangements, embodiments, implementations and equivalents may be employed without departing from the scope hereof as defined by the claims. It is contemplated that the implementation of the components and functions of the present disclosure can be done with any newly arising technology that may replace any of the above-implemented technologies. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What we claim is:

1. A monitoring device for providing a notification to a contact based on a profile configuration associated with a client user comprising:
a memory storing one or more computer-readable instructions; and
a processor configured to execute the one or more computer-readable instructions to:
receive user location data from a client device associated with the client user;
determine a location of the client user based on the user location data;
receive user sensor data from the client device;
determine a status of the client user based on the user sensor data and the location; and
provide the notification to the contact based on the profile configuration, wherein the notification comprises the status.

2. The monitoring device of claim 1, wherein the user location data comprises any of a received signal strength indicator (RSSI), an amplitude of a received signal from the client device, a phase shift of the received signal from the client device, or any combination thereof.

3. The monitoring device of claim 1, wherein the processor is further configured to execute one or more instructions to:
pair the client device with the monitoring device.

4. The monitoring device of claim 1, wherein the user sensor data comprises biometric data associated with the client user.

5. The monitoring device of claim 4, wherein the biometric data comprises any of a movement indicator, a sleep indicator, a blood pressure, a temperature, a pulse, or any combination thereof associated with the client user.

6. The monitoring device of claim 1, wherein the providing the notification comprises the status, the location, or both.

7. The monitoring device of claim 1, wherein the processor is further configured to execute one or more instructions to:
send the user location data and the user sensor data to a monitoring system; and
receive from the monitoring system one or more parameters, wherein determining the location and the status is based on the one or more parameters.

8. A method for providing by a monitoring device a notification to a contact based on a profile configuration associated with a client user, the method comprising:
receiving user location data from a client device associated with the client user;
determining a location of the client user based on the user location data;
receiving user sensor data from the client device;
determining a status of the client user based on the user sensor data and the location; and
providing the notification to the contact based on the profile configuration associated with the client user, wherein the notification comprises the status.

9. The method of claim 8, wherein the user location data comprises any of a received signal strength indicator (RSSI), an amplitude of a received signal from the client device, a phase shift of the received signal from the client device, or any combination thereof.

10. The method of claim 8, further comprising:
pairing the client device with the monitoring device.

11. The method of claim 8, wherein the user sensor data comprises biometric data associated with the client user.

12. The method of claim 11, wherein the biometric data comprises any of a movement indicator, a sleep indicator, a blood pressure, a temperature, a pulse, or any combination thereof associated with the client user.

13. The method of claim 12, wherein the providing the notification comprises the status, the location, or both.

14. The method of claim 8, further comprising:
sending the user location data and the user sensor data to a monitoring system; and
receiving from the monitoring system one or more parameters, wherein determining the location and the status is based on the one or more parameters.

15. A non-transitory computer-readable medium of a monitoring device storing one or more instructions for providing a notification to a contact based on a profile configuration associated with a client user, which when executed by a processor of the monitoring device, cause the monitoring device to perform one or more operations comprising:
receiving user location data from a client device associated with the client user;
determining a location of the client user based on the user location data;
receiving user sensor data from the client device;
determining a status of the client user based on the user sensor data and the location; and
providing the notification to the contact based on the profile configuration, wherein the notification comprises the status.

16. The non-transitory computer-readable medium of claim 15, wherein at least one of:
the providing the notification comprises the status, the location, or both; and
the user location data comprises any of a received signal strength indicator (RSSI), an amplitude of a received signal from the client device, a phase shift of the received signal from the client device, or any combination thereof.

17. The non-transitory computer-readable medium of claim 15, wherein the one or more instructions when executed by the processor further cause the monitoring device to perform the one or more further operations comprising:
pairing the client device with the monitoring device.

18. The non-transitory computer-readable medium of claim 15, wherein the user sensor data comprises biometric data associated with the client user.

19. The non-transitory computer-readable medium of claim 18, wherein the biometric data comprises any of a movement indicator, a sleep indicator, a blood pressure, a temperature, a pulse, or any combination thereof associated with the client user.

20. The non-transitory computer-readable medium of claim 15, wherein the one or more instructions when executed by the processor further cause the monitoring device to perform one or more further operations comprising:
sending the user location data and the user sensor data to a monitoring system; and
receiving from the monitoring system one or more parameters, wherein determining the location and the status is based on the one or more parameters.
